(19) United States
(12) Patent Application Publication
(10) Pub. No.: US 2014/0107558 A1
(43) Pub. Date: Apr. 17, 2014

(54) LAPAROSCOPIC DELIVERY DEVICE

(71) Applicant: Next Wave Surgical LLC, Pompano Beach, FL (US)

(72) Inventors: Ricardo Alexander Gomez, Plantation, FL (US); Sandy Lawrence Heck, Los Angeles, CA (US); Eric William Conley, South Berwick, ME (US); Tristram Ian Jordan, Fort Lauderdale, FL (US); Robert Drew Fanelli, Sayre, PA (US)

(73) Assignee: New Wave Surgical, Pompano Beach, FL (US)

(21) Appl. No.: 14/056,366

(22) Filed: Oct. 17, 2013

Related U.S. Application Data
(60) Provisional application No. 61/714,791, filed on Oct. 17, 2012.
(60) Provisional application No. 61/714,793, filed on Oct. 17, 2012.
(60) Provisional application No. 61/832,489, filed on Jun. 7, 2013.

(51) Int. Cl. A61F 13/26; A61M 31/00

(52) U.S. Cl. CPC A61F 13/263; A61M 31/007
USPC 604/11; 604/60

(57) ABSTRACT

The device for laparoscopically delivering a solid material through a trocar into a body cavity comprises at least: a hollow tube defining a void therein and having a hub at a proximal end thereof, the hub having a proximal opening therein which forms a continuation of the hollow tube; the hollow tube having a prepackaged solid material therewithin; the tube having an outer diameter less than an inner diameter of a trocar through which the tube is inserted into a body cavity, the tube extending past a valve in the trocar when positioned therewithin; the hub having a diameter greater than the inner diameter of the trocar so as not to pass therethrough; and the material being suitably anchored by a string to the device so as not to become lost within the body cavity after being pushed out of the tube.

### CROSS REFERENCE TO RELATED APPLICATIONS

This application claims priority from provisional application Ser. No. 61/714,791 filed Oct. 17, 2012 and entitled Laparoscopic Delivery Device and from provisional application Ser. No. 61/714,793 filed Oct. 17, 2012 and entitled Laparoscopic Absorbent Pad and from provisional application Ser. No. 61/832,489 filed Jun. 7, 2013 and entitled Multifunctional Laparoscopic Delivery Device, the teachings of which are incorporated in their entirety by reference.

### BACKGROUND OF THE INVENTION

This invention relates to a delivery device for dispensing materials through a trocar during laparoscopic or endoscopic procedures which rely on use of trocars inserted through small incisions created in the body, primarily allowing access to the interior of the abdominal region. Such trocars are of small diameter, making it particularly difficult to introduce pieces of solid material into the body cavity. Thus there exists a need for devices that are especially well suited for use with small incisions to assist in the introduction of absorbent gauze, pads, cottonoid, fibrous, mesh or other solid materials. A further requirement is the need of a delivery device with a hub or housing which acts as an anchoring mechanism that can prevent the solid material from becoming trapped in the body of the patient. Still further is the need of a material packaging device that is able to provide an important improvement in the delivery of solid materials.

### PRIOR ART

A delivery device for inserting a surgical mesh is described in United States Patent Publication US 2004/0092970. The delivery device contains a rolled mesh material that is formed into a cylindrical shape and contained within an outer tubular sleeve. The rolled mesh material has a string attachment for removing the mesh material from the outer tubular sleeve. After the delivery device is pushed through the trocar cannula the free end of the string is grasped with a laparoscopic grasping tool and pulled into the abdomen. First, the housing of this delivery device is not effective at preventing the accidental insertion of the complete delivery device into the abdomen, in that the delivery device has an outer diameter smaller than the inner diameter of a trocar cannula. The lack of a way to provide a stop for the delivery device allows for the delivery device to possibly fall into the abdomen. Thus, this prior delivery device increases the potential for complications during the surgery as well as the time required for completion of the surgery. We have found that the duration of the surgery is increased because the positioning of the delivery device is not constrained. Consequently, the surgeon must find the delivery device, grasp it, and then remove it from within the abdomen.

Other delivery devices for laparoscopic surgery include a device, U.S. Pat. No. 5,545,169, consisting of an outer housing with a special size and shape adapted for insertion into a trocar. The housing ends in an elongated tip portion which forms the exit of the delivery device. The housing of the delivery device is hollow for the storage of solid material within its central space. The laparoscopic delivery device dispenses rigid or flexible solid materials. During a surgical procedure, the solid material is inserted into the abdominal cavity of a patient. The laparoscopic delivery device elongated tip portion fits into a trocar and pierces the valve of the trocar. Since the device pierces the trocar valve, a sealing mechanism referred to as the magazine, prevents the loss of insufflation pressure from the insufflated abdomen. In one embodiment, the magazine portion of the delivery device hub or housing is composed of a flexible and collapsible material which forms a seal around the shaft of a laparoscopic tool. The laparoscopic tool can pass into the abdomen of the patient while the delivery device remains in the trocar cannula. The laparoscopic tool must be used to hold the solid material (such as fabric) as well as to position the material within the abdomen. The delivery device embodiment with the flexible magazine design does not prepackage the solid material into a tight space within the delivery device. The lack of prepackaging limits the amount of solid material which can be inserted. In addition, the surgeon must grasp the solid material with a laparoscopic tool and place the solid material into the delivery device. The solid magazine embodiment of this delivery device requires additional trocars and incisions to be made into the body, increasing post-operative pain and healing time. The solid magazine also does not allow a laparoscopic tool to be inserted through the delivery device while the delivery device is seated within the trocar. Still further, the surgeon must obtain the solid material separate from the delivery device and place the solid material into the delivery device.

U.S. Pat. No. 5,203,767 discloses another surgical dispensing apparatus. During a surgical procedure, absorbent material is inserted through a laparoscopic port by a grasping instrument. The grasping instrument aids the positioning of the absorbent material within the body. After the absorbent material is positioned within the body, an anchoring disc or ring prevents the absorbent material from further entering the body. However, devices with loose absorbent materials, such as U.S. Pat. No. 5,203,767, are not easily delivered through the trocar or positioned within the body. No completely satisfactory improvement in the packaging of surgical absorbent dispensing devices has been developed.

U.S. Pat. No. 5,263,927 discloses still another apparatus for dispensing surgical packing. The apparatus stores absorbent material in a reel like device attached to a tube. The tube fits into a trocar and pierces the valve of the trocar. This device has limitations to its usefulness in that the device must be removed from the trocar before a laparoscopic instrument, such as a cauterizing tool or laparoscopic grasper can occupy the trocar. Improved laparoscopic or endoscopic material dispensing devices which facilitate the insertion of material into a body cavity are thus desirable.

### SUMMARY OF THE INVENTION

According to the invention there is provided a device for laparoscopically delivering a solid material through a trocar into a body cavity. The device comprises at least a hollow tube defining a void therein and having a hub at a proximal end thereof. The hub has a proximal opening therein which forms a continuation of the hollow tube and the hollow tube in turn has a prepackaged solid material therewithin. The tube has an outer diameter that is less than an inner diameter of a trocar through which the tube is inserted into a body cavity. During use, the tube extends past a valve in the trocar when positioned therewithin. The hub has a diameter greater than the inner diameter of the trocar so as not to pass therethrough and the solid material is suitably anchored by a string to the device so as not to become lost within the body cavity after being pushed out of the tube.

### BRIEF DESCRIPTION OF THE DRAWINGS

The foregoing features, objects and advantages of the invention will become apparent to those skilled in the art from the following detailed description of a preferred embodiment especially when considered in conjunction with the accompanying drawings in which like numerals in the several views refer to corresponding parts.

FIG. 1A provides a perspective view of a first embodiment of the laparoscopic delivery device constructed in accordance with the teachings of the present invention.

FIG. 1B provides a longitudinal cross sectional view through the laparoscopic delivery device of FIG. 1A.

FIG. 2A provides a perspective view of an alternative embodiment of the laparoscopic delivery device.

FIG. 28 provides a longitudinal cross sectional view through the laparoscopic delivery device of FIG. 2A.

FIG. 3A provides a longitudinal cross sectional view showing the laparoscopic delivery device as inserted into a trocar.

FIG. 3B provides a perspective view showing the laparoscopic delivery device dispensing an exemplary absorbent pad to a target site through a trocar.

FIG. 4A provides a perspective view of a further embodiment of the laparoscopic delivery device.

FIG. 4B provides a longitudinal cross sectional view of the embodiment of FIG. 4A.

FIG. 5A provides a perspective side view of one preferred embodiment.

FIG. 5B provides is a top planar view of the embodiment of FIG. 5A.

FIG. 5C provides a perspective illustration of the device in use with a plunger.

FIG. 5E provides a perspective view of another alternate embodiment.

FIG. 5F illustrates a top planar view of the alternative embodiment, including a spool for storing, attaching, and dispensing a thread or tether.

FIG. 5G provides a planar view of the internal proximal parts of the embodiment of FIG. 5E.

FIG. 6A provides a perspective view of the medical delivery device.

FIG. 6B provides the sectional view of the dispensing device of the preferred embodiment prior to inserting an absorbent material.

FIG. 6C provides a perspective view of the preferred embodiment inserting an absorbent pad to a target site through a trocar.

FIG. 7A-7B illustrate a dispensing device with plunger, the plunger being integrated or provided as a separate structure.

FIGS. 8A-8C illustrate an embodiment including an alignment lock for preventing premature activation of the dispensing device.

FIG. 9A illustrates a perspective view of an alternative embodiment wherein the hub of the device comprises an elongate shroud.

FIG. 9B illustrates an exploded view of the embodiment of FIG. 9A.

FIG. 9C provides a longitudinal cross sectional view through the embodiment of FIG. 9A.

FIG. 10 illustrates a further embodiment of the plunger of the dispensing device.

### DESCRIPTION OF THE PREFERRED EMBODIMENT

The following detailed description is merely exemplary in nature and is not intended to limit the described embodiments or the application and uses of the described embodiments.

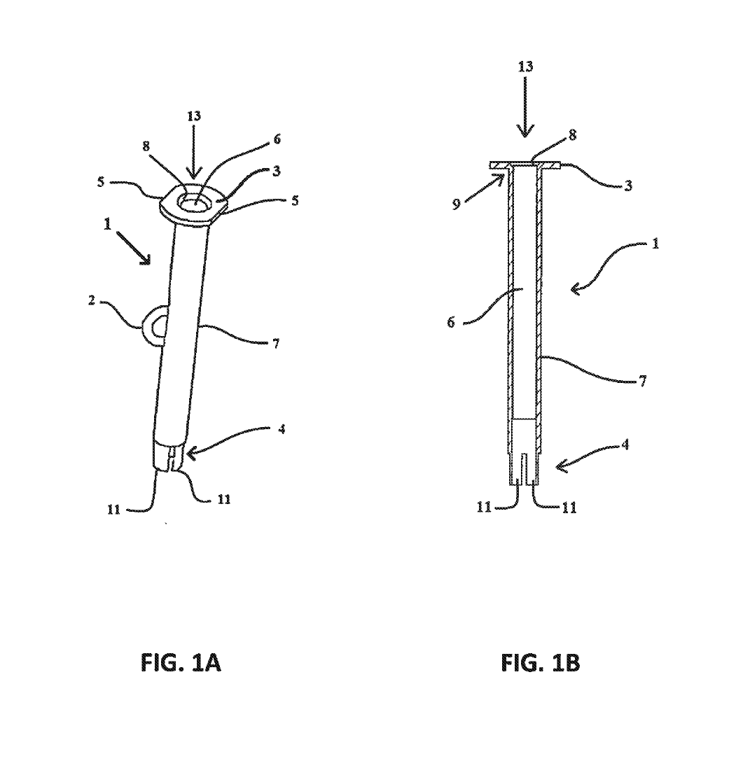

As used herein, the word “exemplary” or “illustrative” means “serving as an example or illustration,” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, terms “upper”, “lower”, “left”, “rear”, “right”, “front”, “vertical”, “horizontal”, and derivatives thereof shall relate to the invention as oriented in FIG. 1.

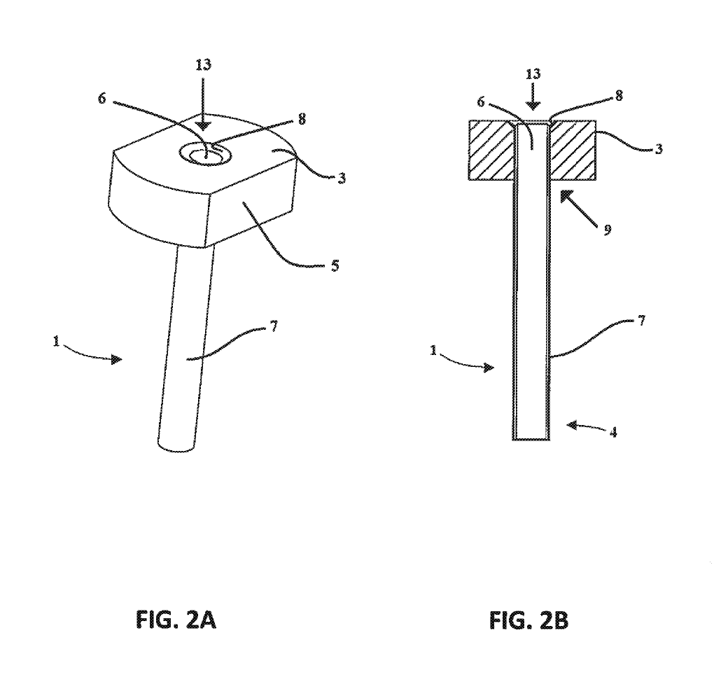

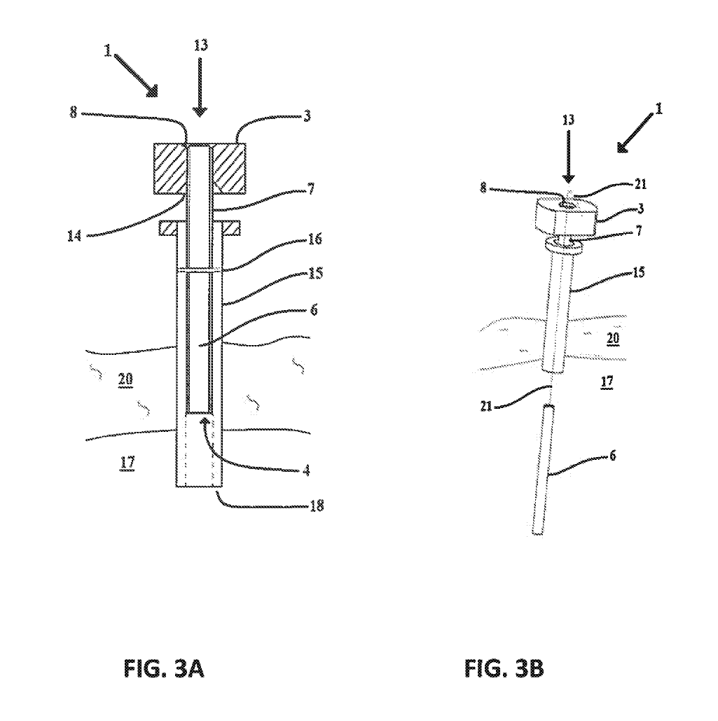

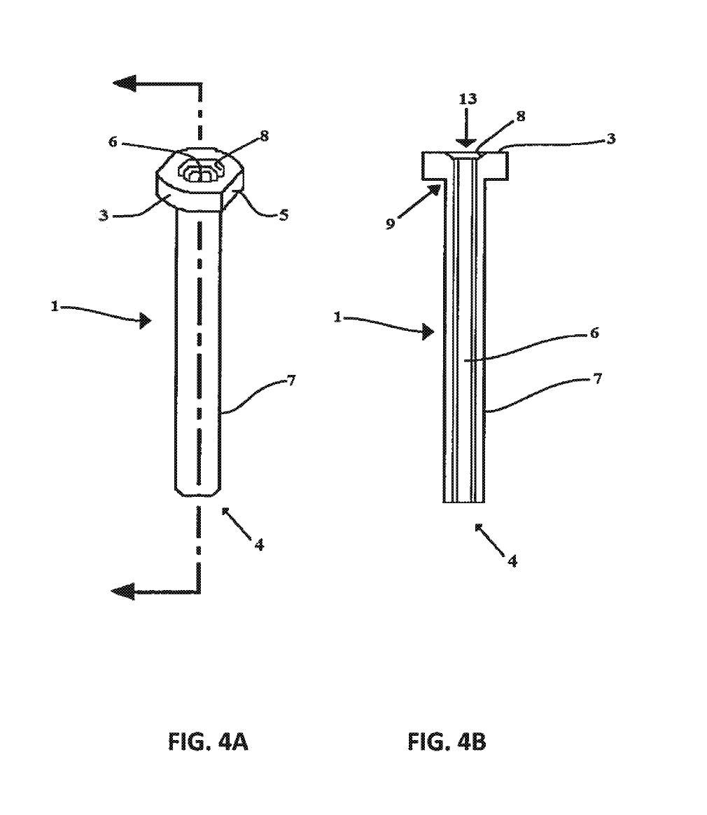

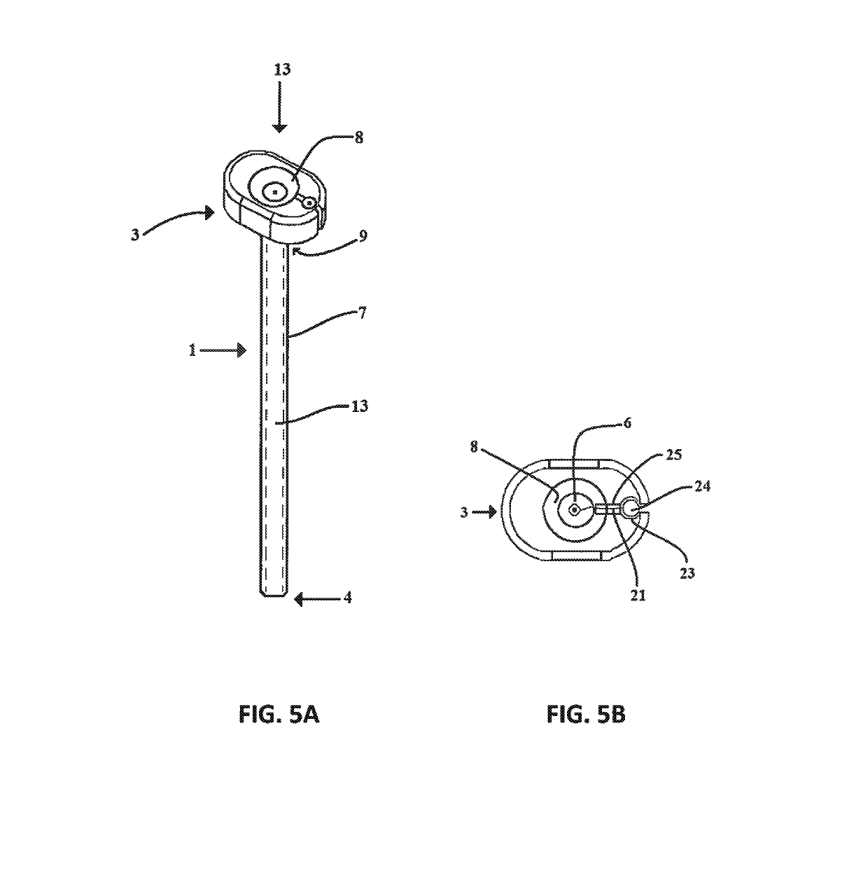

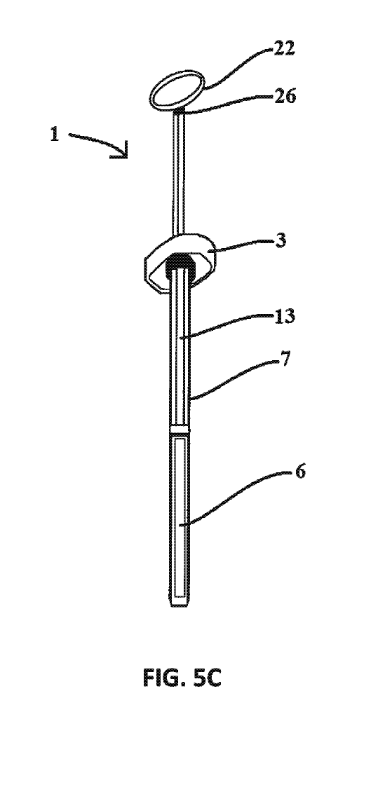

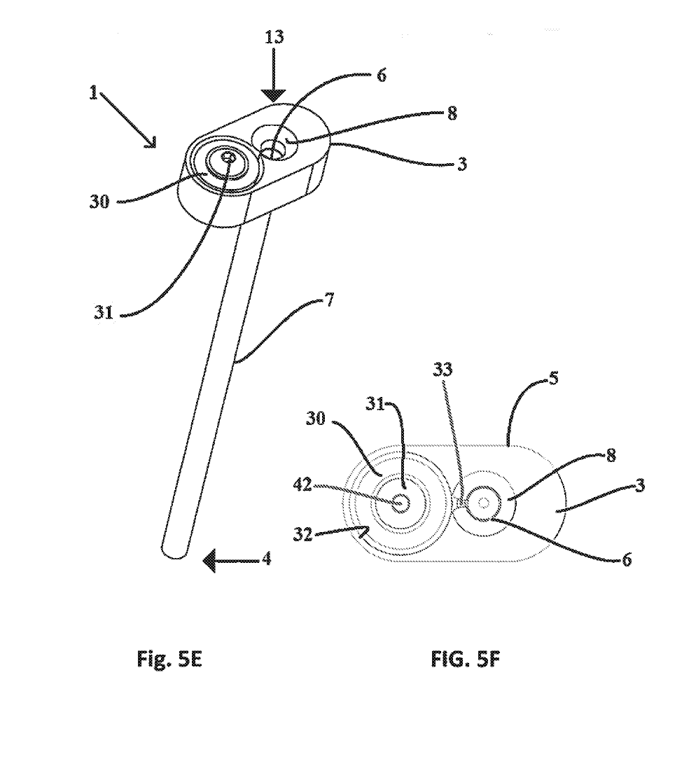

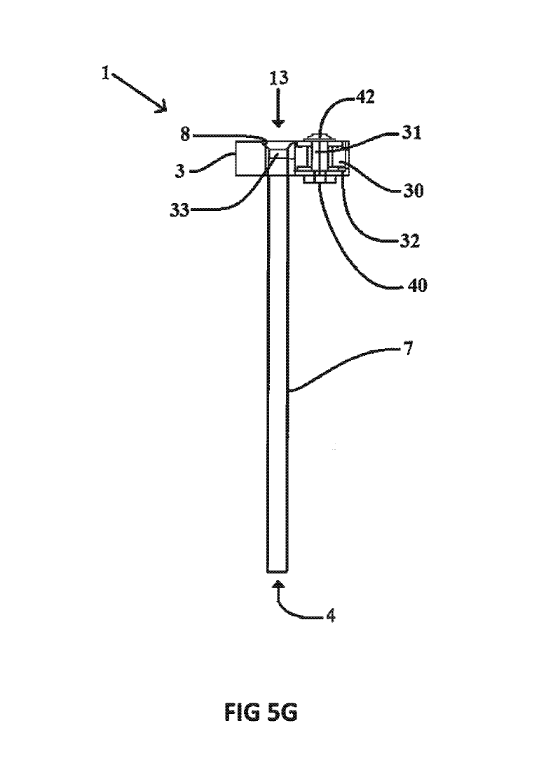

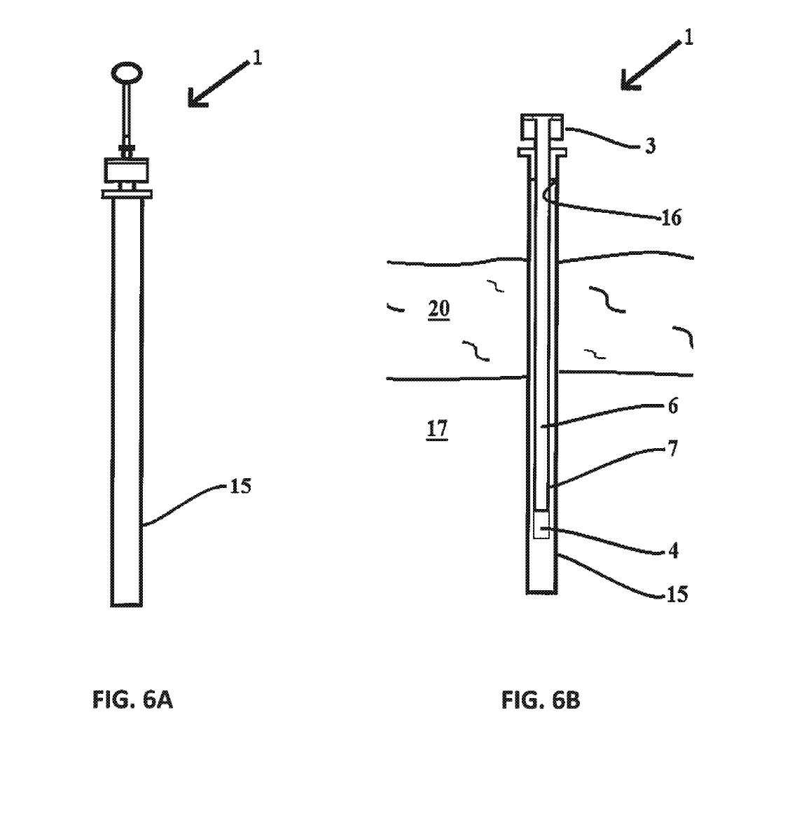

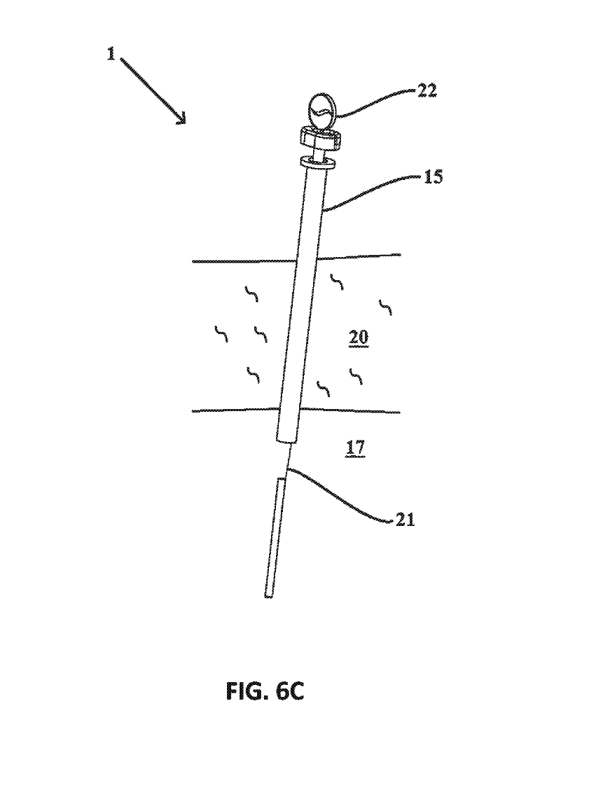

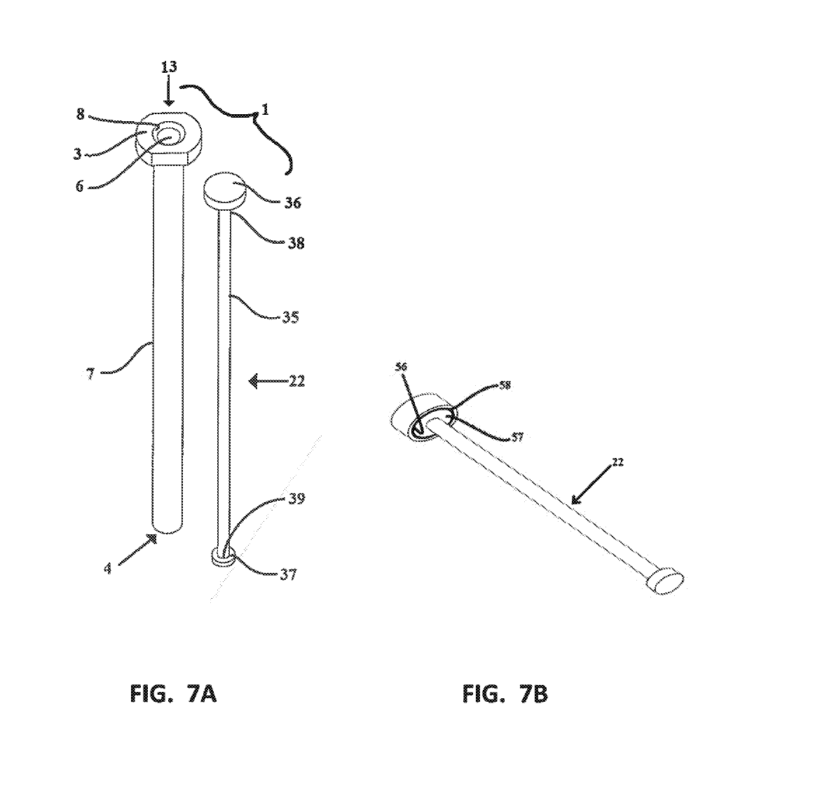

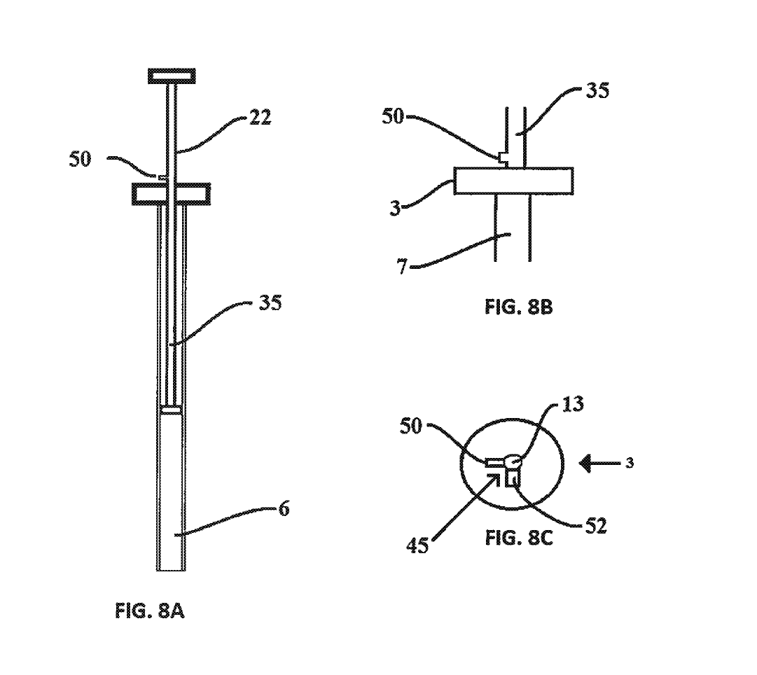

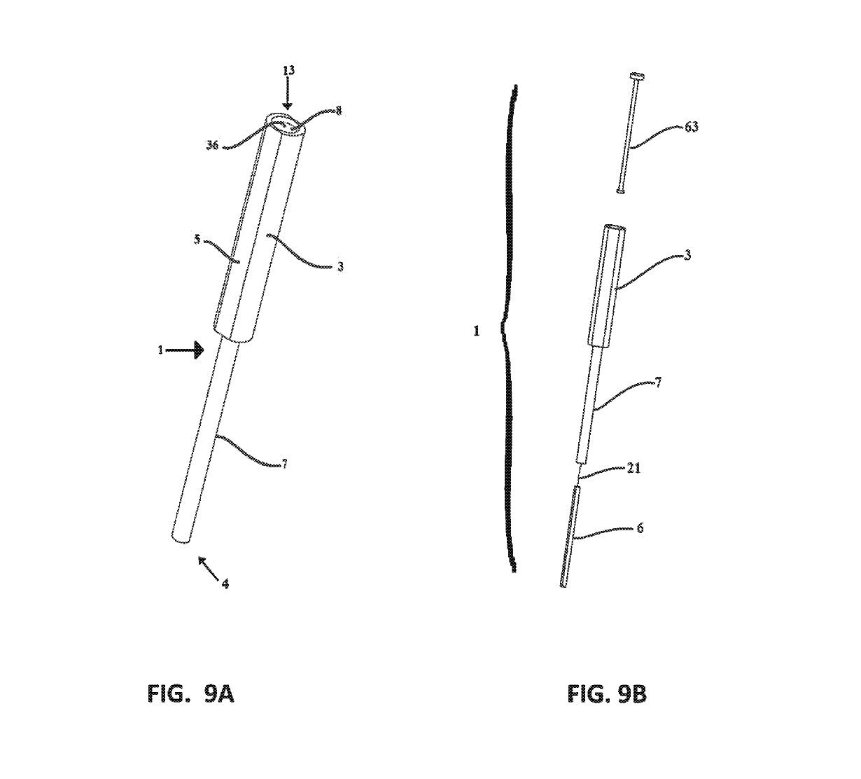

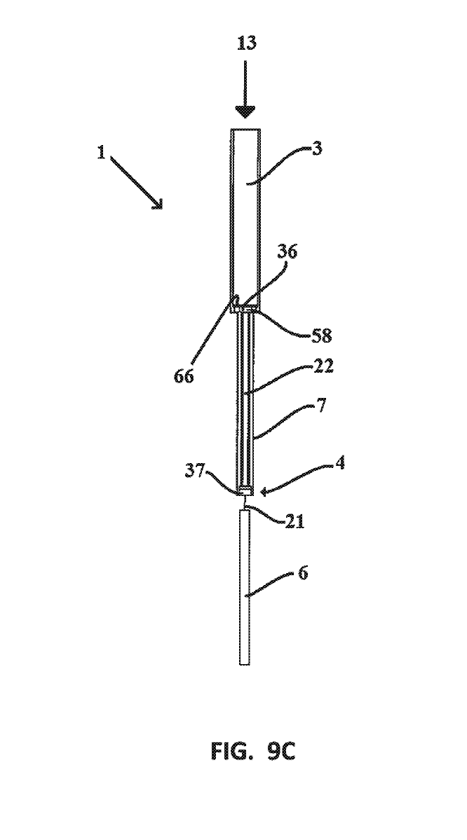

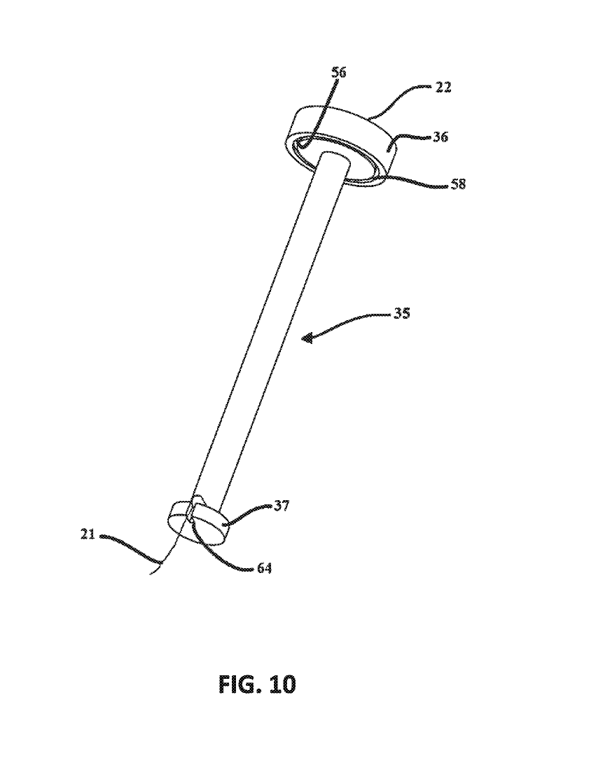

Referring to FIGS. 1A and 1B, a first embodiment of a laparoscopic delivery device 1 is illustrated to comprise an elongate hollow tube or tube portion 7 with a proximal hollow housing or hub 3 at a proximal end 9 thereof, the hub 3 including antiroll portions 5. The delivery device 1 also includes a distal tip portion 4. An outer diameter of the hub 3 is larger than an inner diameter of a standard trocar 15 (FIG. 2A). Standard trocars 15 for laparoscopic surgery may be 5 mm or 10 mm in inner diameter. The antiroll portions 5 define opposite flat surfaces 5 on the hub 3. The flat surfaces 5 prevent the delivery device 1 from rolling off a surgical table or other flat surface (not shown). The housing 3 of delivery device 1 also includes a guide portion 8, which is preferably countersunk or chamfered as shown. The guide portion S acts as a funnel 8, assisting the surgeon in finding the entrance to delivery device 1 during surgery.

The tip portion 4 of the delivery device 1 is sized and configured to fit snugly within the inner diameter of standard trocars as defined above. The tip portion 4 is flexible, comprising prongs 11 of thinner material, relative to the thickness of the material of the tube portion 7. The tip portion 4 comprises two or more prongs 11 in this embodiment. The delivery device 1 may include a loop 2 for attaching solid material 6 being delivered into a body cavity to the delivery device 1 with a radiopaque thread or string 21 (FIG. 3B). The hub 3 of delivery device 1 may also serve as an anchor for the device 1 and prevent the delivery device 1 from accidentally slipping into the body cavity through the trocar 15. Once deployed, the string travels through the trocar, connecting the solid material 6 in the abdomen to the hollow tube outside the body, thereby allowing for easy determination of whether a pad is in the abdomen, easy identification of how many pads are in the abdomen, and easy removal of the pad at the end of the surgical procedure. Since the prepackaged tube housing cannot fit entirely through the trocar, it also acts as an anchoring element which prevents the absorbent pad and string from becoming lost inside the body cavity 17.

The outer diameter of the anchor is preferably greater than 14 millimeters though this should not be construed as limiting, since diameters between 12 millimeters and 14 millimeters, for example, are possible. An absorbent substance or solid absorbent material 6 or pad of the current invention can be square, rectangular, oval, or any other shape. The absorbent material is generally flexible and can be folded, coiled, or rolled in order to maximize the amount of absorbent material contained in a package dispensing system. The absorbent material is generally composed of a soft cotton fabric. The absorbent pads may also incorporate an x-ray detectable element. The absorbent material is sterilized and can be impregnated with an active substance or homeostatic agent. The absorbent pad shown is coiled and compressed to fit into the surgical packaging dispenser to maximize the amount of absorbent pad that can be dispensed from one surgical packaging dispensing device 1.

It will be understood that the elongated tube portion 7 is of a length such that, when the delivery device 1 is inserted into a trocar 15 the distal end 4 of the elongated tube 7 extends past a valve 16 (FIG. 3A) in the trocar 15. The extension of the distal end 4 of the elongated tube 7 past the trocar valve 16 prevents the solid material 6 from becoming caught in the trocar valve 16 when the material 6 is being pushed into the body cavity. The inner diameter of the elongated tube portion 7 is also preferably greater than an outer diameter of standard laparoscopic tool shafts, allowing for passage of laparoscopic tools therethrough.

The hollow elongated tube portion 7 in conjunction with the hollow hub 3 creates a void within which solid material for use within the body cavity is positioned and contained. Passage of any suitable laparoscopic tool (not shown) through the delivery device 1 will push the material 6 into the body cavity.

Turning now to FIGS. 2A-3B, there is illustrated therein a further embodiment of the delivery device 1. The housing 3 of the delivery device 1 is engaged to the elongated tube portion 7 by flaring a proximal end 9 of the tube 7 into the material of the hub 3. The proximal end 9 of the elongated tube 7 here also takes the shape of and provides a funneled guide member 8 for the delivery device 1. A distal end or tip 4 of the hollow elongated tube 7 here also provides an exit for solid material 6 contained within the cavity therein, as best shown in FIG. 3B. In the process of deforming the proximal end 9 of the elongated hollow tube 7, the housing 3 and the elongated tube portion 7 are joined, creating the delivery device 1.

The delivery device 1 also includes a void therewithin for solid material 6 which extends from the proximal end 9 to the distal end 4 of the tube 7. The void or space for receiving solid material 6 may be altered to increase the amount of solid material 6 capable of being packed within the elongated tube portion 7. The solid material 6 may be inserted into the elongated tube portion 7 from either end of tubing 7 of the delivery device 1. This embodiment is similarly configured to allow passage of laparoscopic tools therethrough in like fashion to the previous embodiment and thus will not be reiterated here. This device 1 further includes a countersunk distal edge 14 to the void in the hub 3 to allow the elongated tube portion 7 to be easily inserted into the housing 3 for engagement thereto. The elongated tube portion 7 is preferably made from a thin plastic material. The elongated tube 7 and hub 3 could also be made of other thin materials, such as stainless steel alloys, metal foils or plastics.

With specific reference to FIGS. 3A and 3B, they depict use of both embodiments presented herein so far during surgery. As illustrated, the delivery device 1 is inserted into trocar 15 to a point where distal tube end 4 extends past trocar valve 16. The distal end 18 of trocar 15 is inserted into, for example, cavity 17 and past abdominal wall 20. Once so positioned, a laparoscopic instrument or tool (not shown) would be inserted through the funnel or guide portion 8 of the delivery device 1 to push the solid material 6 out past the distal end 18 of the trocar 15.

The proximal enlarged housing piece would be made of plastic in the preferred embodiment, but can be manufactured from other materials. In the preferred embodiment, the enlarged proximal housing and the elongated tube would be two separate pieces joined together, but they could also be manufactured as a single piece.

During surgery, a laparoscopic instrument enters the funnel portion 8 of the surgical packaging dispenser is pushed towards the end of the trocar. Absorbent pad 6 is then pushed along the tube and dispensed into the abdomen 17 of the patient. Once inside the body, the absorbent pad would unfurl and could be positioned near bleeding sites or wherever the pad or material 6 is required using, for example, a laparoscopic grasper (not shown). The absorbent pad or material 6 may also be attached to the hub 3 of the delivery device 1 as best illustrated in FIG. 3B.

With reference to FIGS. 4A-4B, a further embodiment of a laparoscopic dispensing device 1 is seen to comprise a hollow hub or housing 3, which also serves as an anchor, as will be described in detail hereinafter, and a hollow elongate tube portion 7. The hub or housing 3 includes antiroll surfaces 5, and a countersunk guide section 8 surrounding a proximal opening 13. The elongate tube portion 7 may be either joined to or integrally formed with the hub 3 to create the dispensing device 1. The enlarged diameter of the hub 3 is larger than the inner diameter of a standard trocar. The enlarged diameter of the hub 3 prevents the dispensing device 1 from falling into the abdomen through the trocar. The enlarged diameter is preferably larger than 14 millimeters. However, diameters between 12 millimeters and 14 millimeters are also possible to use. The diameter of the elongate tube portion 7 may be varied so that it fits snugly inside a range of different sized standard trocars for laparoscopic surgery including 5 mm to 12 mm in diameter embodiments. There are two antiroll surfaces 5 provided opposite each other along a periphery of the hub 3. The antiroll surfaces 5 are flat and prevent the dispensing device 1 from rolling off a surgical table or other flat surface.

Positioned within the hollow interior of the dispensing device 1 is a material pad 6. The pad 6 may be an absorbent pad and can have a variety of different dimensions which may be accommodated within the hollow dispensing device 1. The pad 6 should not, however, be longer than the dispensing device 1 in order to prevent the pad 6 from extending out of either the hub 3 or distal end 4 of the dispensing device 1. The pad 6 can be folded, rolled, coiled or packaged in another manner into the dispensing device to maximize the amount of material that can be dispensed from one dispensing device 1. The dispensing device 1 includes a guide surface 8 for guiding a laparoscopic tool such as a grasper, cauterizing instrument, or other laparoscopic tool (not shown) into the proximal opening 13 and through the hollow interior of the dispensing device 1. Alternatively, in the case where a laparoscopic tool cannot fit through the device 1, an appropriately sized plunger 22 may be provided. The guide portion 8 acts as a funnel to help the surgeon find the proximal opening 13 of the dispensing device 1 during surgery. Once a tool or the plunger 22 is inserted into the proximal opening 13, the surgeon uses the tool or plunger 22 to push the absorbent pad or other material 6 into the abdominal cavity 17 of the patient. A thread or string 21 (FIG. 3B) for attaching the absorbent pad 6 to the dispensing device 1 is attached to the absorbent pad 6 and a free end of the thread 21 is attached to the dispensing device 1. The thread 21 may, for example, attach to the dispensing device 1 by a pin (not shown) inserted into an opening (not shown) in the hub 3 of the dispensing device 1. Alternatively, the thread 21 may attach to a spool, loop, slot, or hook (not shown) in the hub or housing 3. The thread 21 can be tied, sewn, or glued to the spool, loop, slot, hook, or opening. The thread 21 is long enough to facilitate placement of the absorbent pad 6 at desired locations within the abdominal cavity 17.

Referring to FIG. 4B, the figure provides a longitudinal cross sectional view through the dispensing device 1 of FIG. 4A. The material 6 is clearly seen to be contained within the central void of the hollow dispensing device 1 in this figure.

In FIG. 5A is shown a preferred embodiment of the dispensing device 1. The dispensing device 1 has an elongated tube portion 7. The distal end 4 of the elongated tube portion 7 provides an exit for the material (not shown). The proximal end 9 of the elongated tube portion 7 is joined to a hub or housing 3. The elongated tube portion 7 is long enough, such that, when the dispensing device 1 is inserted into a trocar 15, the distal end 4 of the elongated tube 7 extends past trocar valve 16 (FIG. 6B). The extension of the distal end 4 of the elongated tube 7 past the trocar valve 16 prevents the material within the tube 7 from becoming caught in the trocar valve 16 as it is pushed into the body cavity 17. The housing or hub 3 of the dispensing device 1 also acts as an anchoring mechanism for preventing the dispensing device 1 from entering the body through the trocar 15. The horizontal extent of the housing 3 is larger than the inner diameter of the trocar 15 into which the dispensing device is inserted. The housing or hub 3 of the dispensing device may also receive a pin (not shown) for attaching the thread, string, or tether 21 of the absorbent pad or material 6 to the dispensing device 1 to prevent the material from becoming lost within the body cavity 17 and allows the surgeon to easily locate the material 6 when it is to be removed. A plunger 22 (FIG. 7A) is provided with the dispensing device 1 for pushing the material out of the device 1.

The tube 7 comprises a hollow instrument receiving portion comprising a proximal opening 13 leading to the void therein, with a funneled entrance guide 8. The proximal opening 13 is sized to receive standard laparoscopic tools, such as, a laparoscopic grasper, cauterizing instrument, other laparoscopic tool, there through. In case a laparoscopic device cannot fit, an appropriately sized plunger 22 (FIG. 7B) is provided. The funneled entrance has a tapered or conical cross section that guides a cauterizing tool or other laparoscopic tool into the central opening 13.

FIG. 5B provides a top view of the preferred embodiment of the dispensing device 1. The housing or hub 3 of the dispensing device 1 is attached to the elongated tube portion 7 to create the dispensing device 1. The hub 3 has a funneled guide portion 8 for guiding laparoscopic tools into the central opening 13 of the dispensing device 1. The central opening 13 of the dispensing device 1 leads to a void within the tube 7 within which is stored material 6 to be dispensed from the device 1.

In addition, the housing 3 contains an opening 23 for receiving a pin 24. The pin 24 prevents the material 6 from separating from the dispensing device 1 by engaging the thread 21 attached to the material 6 within the opening 23. The housing 3 also has a smooth cutout 25 for guiding a thread, tether, or string 21 from the central opening 13 to the housing opening 23 which receives the pin 24, minimizing potential damage to the string, thread, or tether 21.

The elongated tube portion 7 and the housing 3 of the dispensing device 1 may be made from plastics, metals, or composite materials. The elongated tube portion 7 can also be manufactured from other thin materials such as metal foils. The elongated tube portion 7 wall thickness can vary depending on the material used. When constructed from stainless steel, for example, the elongated tube portion 7 can have a very thin wall; as thin as 0.004 inches.

FIG. 5C, shows dispensing device 1 in use. The plunger 22 is inserted into the dispensing device 1 via central opening 13 and material 6 is pushed out of the dispensing device 1 by the plunger 22. Any excess thread 21 is looped along the length of the material 6 within the void in the elongated tube 7. When the material 6 is pushed into the body cavity 17 (FIG. 6B), the excess thread unloops. The dispensing device also has a sealing mechanism 26 for preventing the escape of gas from the insufflated abdomen. Preferably sealing mechanism 26 is a ring located on the shaft of the plunger. When ring 26 makes contact with the inner diameter of the dispensing device elongated tube 7, a sealing effect is created. The ring 26 can be made of a rubber compound or other material which deforms.

A spool 30 distinguishes the embodiment of FIG. 5E from the previous one. The spool 30 replaces pin 24 of FIGS. 5A-5C and acts to retain, store, and dispense thread or tether (not shown). Spool 30 rotates around shaft 31 to dispense thread or tether. The thread or tether 21 is engaged around the central shaft 31 of the spool 30, such as by forming a knot in one end of the thread or tether. The knot securely connects the material 6 to the dispensing device 1 and prevents the absorbent pad from becoming lost within the body. Thread or tether 21, is coiled around the central shaft 31 of the spool 30 until the excess thread or tether is completely contained by the spool 30. The thread or tether is dispensed from the dispensing device 1 by pressing material 6 out of the dispensing device 1 or, once the material 6 is within the body cavity 17, by pulling material 6 with a laparoscopic grasper. Housing 3 contains spool port 32 for receiving spool 30. Spool port 32 has a larger diameter than spool 30 so that the spool 30 can freely spin around shaft 31.

FIG. 5F provides a top view of the hub 3 which includes port 32 for mounting spool 30 and shaft 31. Housing 3 also has a cutout 33 for guiding thread or tether (not shown) into the spool. Cutout 33 is aligned with the axis of the spool shaft 31 and the proximal opening 13 of the dispensing device 1.

FIG. 5G provides a partially cut away side view of the dispensing device 1. Spool 30, shaft 31, port 32, and cutout 33 are seen. Housing 3 includes a shaft attachment 40. Attachment portion 40 is an extension of the lower housing 3 surface. When the shaft 31 is attached using a shoulder screw type fastener 42, the shaft attachment portion 40 has a threaded opening (not shown) for threading the shoulder screw 42 into the dispensing device housing extension 40.

FIG. 6A shows the dispensing device 1 inserted into trocar 15 for use. Dispensing device housing 3 is seen above the top of the trocar 15 and is larger in diameter than the inner diameter of the trocar 15 to prevent the dispensing device 1 from entering the body through the trocar 15. The dispensing device distal exit 4 is positioned past trocar valve 16 and close to or within abdominal cavity 17. The location of the dispensing device exit 4 past the trocar valve 16 prevents the material 6 from becoming stuck in the trocar valve 16 after exiting the dispensing device 1. The trocar valve 16 rests against the outer surface of the dispensing device tube 7 and seals around the outside of the dispensing device tube 7 to prevent loss of insufflation pressure from the abdomen. During surgery, the provided plunger 22 (FIG. 6C) is inserted into the dispensing device housing 3 and pressed toward the abdominal space 17, dispensing the material 6. Once inside the body cavity 17, the material 6 is positioned as desired, such as, for example, when comprising an absorbent pad, near bleeding sites needing an absorptive material.

FIG. 6B shows dispensing device 1 deployed through abdominal wall 20 via trocar 15. The dispensing device is inserted into trocar 15. Once within the body cavity 17, the material may be unpacked and positioned at a desired site with a laparoscopic grasper (not shown). The material 6 is attached to the hub or housing 3 as described above. The thread, string, or tether 21 may be radiopaque for x-ray detectability. The material 6 may also include a radiopaque strip, tape or stitching that allows for identification if it is left within the body cavity 17.

FIG. 7A is a depiction of another embodiment of dispensing device 1. Plunger 22 as shown here includes a central shaft 35 with enlarged portions 36 and 37, respectively, on proximal and distal ends 38 and 39 of the shaft 35. The plunger 22 is inserted into the integrated dispensing device 1 via central opening 13. The distal end 39 of the plunger 22 is oriented toward the distal end 4 of the dispensing device 1, and contacts the prepackaged material (not shown) to push it out of the dispensing device 1. The plunger 22 is of sufficient length to push the prepackaged material completely out of the dispensing device 1. The thread, string, or tether (not shown) may also attach the material to either the proximal 36 enlarged portion, the shaft 35, or the distal enlarged portion 37 of the plunger 22, or to the hub 3 of the dispensing device 1 by pinning as previously described or can be tied, glued, etc. to the dispensing device 1. The proximal enlarged portion 36 of the plunger 22 is larger than the proximal opening 13 in the dispensing housing to prevent the plunger 22 from entering the body cavity 17 it will be understood in regard to this embodiment that the plunger 22 may be loose or integrated into the device 1, as an example.

FIG. 7B provides a perspective view of the plunger showing a recess 56 on an underside 57 of the proximal enlarged portion 36 for receiving a seal element 58 therein. When the plunger 22 is fully deployed, the seal element 58 makes contact with the upper surface of the housing 3 to prevent insufflation gas from escaping the body cavity 17. Alternatively, the seal element 58 could be placed on the distal enlarged portion 37 to maintain sealing during the dispensing of the absorbent material 6.

FIGS. 8A-8C provide a detailed view of another embodiment including an alignment lock 45. The alignment lock 45 prevents plunger 22 from dispensing material 6 prematurely. The lock 45 comprises a protrusion 50 at the distal end 39 of plunger shaft 35 and a cutout 52 formed in the hub 3. The cutout 52 allows protrusion 50 to enter the dispensing device 1 when properly aligned therewith. The locking mechanism 45 thus prevents the premature activation. The plunger 22, once aligned can be pushed completely into the dispensing device 1, causing the material 6 to be dispensed. Other variations of the plunger lock 45 may also be proposed. It will be understood that the shaft 35 of the plunger 22 with the protrusion 50 thereon is sized to be accommodated by the tube portion 7.

With reference to FIGS. 9A-9C, a further embodiment of the dispensing device 1 is shown. This dispensing device 1 also comprises hollow tube portion 7 containing an absorbent pad (not shown) and a plunger 22 integrated within the now elongated housing or hub 3, which creates a protective shroud for the plunger 22. Shrouding of the plunger 22 within the housing 3 prevents the plunger 22 from accidentally being pushed toward distal end 4. Without the shrouding hub 3, if the plunger 22 were to be pushed toward the distal end 4 of the dispensing device 1 accidentally, material 6 may partially or fully exit the dispensing device 1. Proximal opening 13 in the hub 3 of the dispensing device 1 is larger in diameter than the inner diameter of the void in the hollow tubular portion 7. Because the inner diameter of the hollow tubular portion 7 below the hub 3 is smaller than the diameter of proximal enlarged portion 36 of the plunger 22, the plunger 22 is prevented from being pushed through the dispensing device 1 into the body cavity 17. Activation of the device 1 may be initiated by inserting a laparoscopic grasper, cauterizing instrument, other laparoscopic tool, into the housing 3 via proximal opening 13. If a laparoscopic device cannot fit, another appropriately sized plunger 63 may be used.

FIG. 9B, an exploded view of the dispensing device 1 of FIG. 9A is provided. The plunger 22 of this dispensing device 1 also preferably includes seal element 58 FIG. 9C on an underside of the enlarged proximal portion 36 of the plunger 22.

With reference to FIG. 9C, it provides a longitudinal cross sectional view through this embodiment of the dispensing device 1, after activation. The device is activated by pushing plunger 22 toward the distal end 4 of the dispensing device 1, until enlarged proximal portion 36 thereof abuts an internal distal shoulder 66 of the housing 3. The plunger 22 is pushed down with a laparoscopic grasper, cauterizing instrument, other laparoscopic tool. If a laparoscopic device cannot fit, a second appropriately sized plunger 63 FIG. 9B may be used for activation. Proximal opening 13 of the hub 3 is larger in diameter than the inner diameter of the hollow tube portion 7, retaining the plunger 22 within the dispensing device 1. In order to avoid interference with the seal 58, a thread 21 attachment may be provided on the shaft 35 of the plunger 22 or the distal enlarged portion 37. If the seal element 58 is located on the distal enlarged portion 37 of the plunger 22, the seal element 58 may incorporate thread 21 attachment structure. For example a hole, slot, hook, or loop could be molded into the seal member 58, away from the seal surfaces which make contact with the internal distal shoulder 66 of the dispensing device 1. The seal element 58 can alternatively be located on the enlarged distal portion 37 of the plunger 22 such that the seal member 58 makes contact with the inner surface of the hub 3 of the dispensing device 1, as with the other embodiments having a plunger 22. The advantage of such syringe type seal is the continuous sealing of the dispensing device 1 before, during, and after activation, avoiding loss of insufflation. It will be understood that seal element 58 may comprise an O-ring, flap or any other suitable sealing element.

With reference to FIG. 10, a further embodiment of the plunger 22 is illustrated. This plunger 22 also incorporates the two enlarged end sections 36 and 37 connected by the shaft 35. The plunger 22 also incorporates seal element 58 within recess 56 on the underside of the proximal enlarged end portion 36. A cutout or notch 64 is provided in the distal enlarged portion 37 for engaging string or tether 21 attached to material 6.

Further, utility of the device 1 includes its ability to create hemostasis within the body. The material 6 within the dispensing device 1, when combined with a bioactive agent, such as epinephrine, can stop bleeding at wound sites within the body cavity 17, the material 6 acting as a carrier for the bioactive agent. The first step toward creating hemostasis using the dispensing device 1 is to apply the bioactive agent to the material 6 with the bioactive agent becoming incorporated into material 6. The bioactive agent can be applied to the material 6 while the material 6 is contained within the dispensing device 1 by applying the bioactive agent via the proximal opening 13 of the dispensing device 1.

In the preferred embodiment (FIG. 3A) the bioactive agent is applied by submerging the distal opening 4 of dispensing device 1, into a tray or container (not shown) having the bioactive agent. Thus the absorbent pad 6 absorbs and becomes saturated with the bioactive agent. Due to the absorbent qualities of the pad material 6, the bioactive agent is retained by the absorbent material 6 during subsequent insertion of the device 1 into the body cavity 17 and the absorbent material 6 containing the bioactive agent is dispensed from the delivery device 1. The absorbent material 6 containing the bioactive agent can then be placed on bleed sites within the body cavity 17 to create hemostasis.

It should be noted that the absorbent pad 6 can also be used to clean the lenses of laparoscopic devices.

The above-described embodiments are merely exemplary illustrations set forth for a clear understanding of the principles of the invention. Many variations, combinations, modifications, or equivalents may be substituted for elements thereof without departing from the scope of the invention. Therefore, it is intended that the invention not be limited to the particular embodiments disclosed, but should only be limited as necessitated by the scope of the accompanying claims.

1. A device for laparoscopically delivering a solid material through a trocar into a body cavity comprising at least:
a hollow tube defining a void therein and having a hub at a proximal end thereof; the hub having a proximal opening therein which forms a continuation of the hollow tube;
the hollow tube having a prepackaged solid material therewithin;
the tube having an outer diameter less than an inner diameter of a trocar through which the tube is inserted into a body cavity, the tube extending past a valve in the trocar when positioned therewithin;
the hub having a diameter greater than the inner diameter of the trocar so as not to pass therethrough; and
the material being anchored by a string so as not to become lost within the body cavity after being pushed out of the tube.

2. The device of claim 1 wherein the proximal opening includes a countersunk or chamfered guide portion about a periphery thereof.

3. The device of claim 1 wherein the hub is unitary with the tube.

4. The device of claim 1 wherein the hub is separate from the tube.

5. The device of claim 1 further including a plunger.

6. The device of claim 5 wherein the plunger is separate from the device.

7. The device of claim 6 wherein the plunger is permanently retained within the device.

8. The device of claim 1 wherein the prepackaged solid material is sized and configured to be compactly received within the void defined by the hollow tube.

9. The device of claim 1 wherein the prepackaged solid material is absorbent.

10. The device of claim 1 wherein the prepackaged solid material serves as a carrier for a liquid.

11. The device of claim 10 wherein the liquid is an antibiotic.

12. The device of claim 10 wherein the liquid is a bioactive agent.

13. The device of claim 1 wherein the proximal opening is sized to receive a surgical tool therethrough.

14. The device of claim 1 wherein the material is anchored by a string to the tube.

15. The device of claim 1 wherein the string is anchored to the hub.

16. The device of claim 14 wherein the string is anchored to the hub by a pin.

17. The device of claim 14 wherein the string is anchored to the hub via a spool engaged to the hub.

18. The device of claim 14 wherein the string is anchored to a plunger.

19. The device of claim 5 wherein the plunger has a proximal end portion of greater diameter than the inner diameter of the tube.

20. The device of claim 5 wherein the plunger includes a sealing element for sealing against inner structure of the device when received therein.

21. A solid absorbent surgical pad and tubular laparoscopic delivery device therefor comprising, a pad formed from a water absorbent sterile fibrous material that is folded, coiled or rolled into an elongated rod shaped member and is compressed to a size which is reduced sufficiently to be passed into a tube comprising a body of a delivery device that is sized, constructed and arranged to be at least partially inserted into a trocar and the pad is slidably and removably retained within the delivery device, and
a flexible tether is connected between the pad and the delivery device to prevent the loss of the pad following ejection of the pad from the delivery device into the body of the patient.

22. The surgical pad and delivery device of claim 21 wherein the tether is a string and the pad is connected by the string to a hub that acts as an anchor on the delivery device by preventing the delivery device from passing entirely through the trocar.
